(19) United States
(12) Patent Application Publication
(10) Pub. No.: US 2020/0220193 A1
(43) Pub. Date: Jul. 9, 2020

(54) MICROBIAL METHOD AND APPARATUS OF ELECTRICAL POWER GENERATION

(71) Applicant: NATIONAL CENTRAL UNIVERSITY, Taoyuan City (TW)

(72) Inventor: Chun-Ming Huang, Kaohsiung City (TW)

(21) Appl. No.: 16/732,957

(22) Filed: Jan. 2, 2020

Related U.S. Application Data
(60) Provisional application No. 62/788,076, filed on Jan. 3, 2019.

(51) Int. Cl. H01M 8/16; H01M 4/90; C12N 1/20; C12P 7/40; H01M 4/86

(52) U.S. Cl. CPC H01M 8/16; H01M 4/9008; H01M 4/8626; C12P 7/40; C12N 1/20

(57) ABSTRACT

A method is provided for electrical power generation, including the following steps: (a) obtaining skin bacteria from human skin to isolate an electrogenic bacteria; (b) culturing the electrogenic bacteria in a source medium to form a cultured solution; (c) applying the cultured solution to a microbial fuel cell; and (d) allowing the electrogenic bacteria to ferment in the microbial fuel cell, and to produce butyric acid or butyrate, and thereby to form an electrical current. An apparatus is also configured to perform the specified method, which includes an anode, a cathode, and a proton exchange membrane, and the electrogenic bacteria is cultured in this apparatus to ferment and thereby to generate electrical current.

### CROSS-REFERENCE TO RELATED APPLICATIONS

This application claims the benefit of U.S. Provisional Application No. 62/788,076, filed on Jan. 3, 2019, the content of which are hereby incorporated by reference in their entirety.

### FIELD OF THE INVENTION

The present invention relates to a microbial method and an apparatus for generating electrical power. In particular, the present invention relates to a method of using electrogenic bacteria that are capable of creating an electrical current by fermentation, and thereby relates to the power-generating method and apparatus thereof.

### BACKGROUND OF THE INVENTION

Using microbes to generate electricity was conceived in the early twentieth century. Bacteria have been widely employed in developing microbial fuel cell (MFC), which is a bio-electrochemical system that drives electric current. In general, the electrogenic bacteria catabolize organic substrates, and the catalytic redox activity drives the transfer of electrons (electrical charge) from or to a solid electrode to generate bio electricities.

Most of electrogenic bacteria used in MFCs are isolated from wastewater, anaerobic reactor sludge, and marine sediment and most of them are metal-reducing bacteria, such as Geobacter sulfurreducens, Geobacter metallireducens, Shewanella putrefaciens, Clostridium butyricum, Rhodoferax ferrireducens, and Aeromonas hydrophila. Among these bacteria, G. sulfiurreducens is one of most extensively studied/used bacteria capable of high current densities in MFCs. Geobacter species have been shown to be important in the anaerobic degradation of several carbon sources. However, most Geobacter species are extremely intolerant of oxygen, and technological possibilities are limited. On the other hand, Shewanella oneidensisis able to survive in presence of oxygen, but it cannot completely oxidize the organic substrate typically used (e.g., lactate) in MFCs, leaving electrons unutilized and waste products such as acetate. Therefore, when a MFC system requires a microorganism to completely oxidize the organic substrates to CO₂ and achieve higher columbic efficiencies, the Shewanella species may not be a good option.

With the highly efficient bacterial electron transport system, MFCs using bacteria as biocatalysts usually have high conversion efficiency in harvesting up to 90% of the electrons from the bacterial electron transport system. A single MFC usually produces low power (0.6-0.8V), which is attractive for power generation applications that require only low power without replacing batteries, such as biosensors, bioassays or medical devices. And specific electrogenic bacteria candidates for diverse applications are definitely demanded to elaborate above mentioned advantages.

MFCs involve many substrates in generating electricity, virtually any organic material (carbon source) could be used to feed the MFCs, including carbohydrates, proteins, volatile acids, cellulose and wastewater. Practically, processed wastewater has been widely used to produce bioelectricity in dual- and single-chamber MFCs.

MFCs using bacteria as the biocatalyst usually generate power through a biological process called fermentation. For example, when bacteria consume a substance such as sugar in anaerobic conditions, they produce carbon dioxide, protons/hydrogen ions and electrons, and forms electron transport chain to drive the electrical charge. Other bacteria used in MFCs are able to oxidize acetate, ethanol, lactate, or propionate as carbon/fuel source.

Based on the above mentioned capability of MFCs, people are making lots of efforts to utilize the carbon/fuel sources (e.g., glucose) or even metabolites (e.g., lactate) naturally existing in the human body for generating sustainable electricity, which may supply power for the implantable or diagnostic medical devices. Therefore, a better biologically electrogenic system remains an unmet need.

### SUMMARY OF THE INVENTION

Unless otherwise specified herein, all scientific and technical terms used herein will have the meanings that are commonly understood by the skilled person.

According to the aforementioned unmet needs, the application provides a microbial method of electrical power generation, which comprises the following steps: (a) obtaining skin bacteria from human skin to isolate a species of electrogenic bacteria; (b) culturing the electrogenic bacteria in source medium to form cultured solution; (c) applying the cultured solution to a microbial fuel cell; and (d) allowing the electrogenic bacterium to ferment in the microbial fuel cell, and to produce butyric acid or butyrate to form an electrical current. Therefore, a better biologically electrogenic system is provided for the implantable or diagnostic medical devices.

According to a preferred embodiment of the present invention, the electrogenic bacteria used in the method can be Staphylococcus epidermidis or other bacteria isolated from human microbiome.

According to a preferred embodiment of the present invention, the cultured solution is formed by culturing the electrogenic bacteria in the source medium, and the cultured solution is applied to the anode of the microbial fuel cell to ferment.

According to a preferred embodiment of the present invention, the electrical current is generated by using the method according to the application, and the voltage of the electrical current ranges from 30 mV to 150 mV.

The application provides an apparatus of electrical power generation, which comprises an anode configured to hold electrogenic bacteria, a cathode, and a proton exchange membrane. The electrogenic bacteria is cultured to ferment and therefore to produce butyric acid or butyrate to form electrical current.

According to a preferred embodiment of the present invention, the anode can be a carbon felt.

According to a preferred embodiment of the present invention, the cathode can be a carbon cloth.

### BRIEF DESCRIPTION OF THE DRAWINGS

FIG. 1(a) illustrates graphically the read value at OD562 nm of the fermented supernatant after fermentation in different conditions. M: rich medium; H: H₂O; BS: S. epidermidis bacteria inoculated (10⁵ CFU/ml); and G: glycerol (2%).

FIG. 1(b) illustrates graphically the read value at OD562 nm of the fermented supernatant after fermentation in different conditions. M: rich medium; H: H₂O; BP: P. acnes bacteria inoculated (10⁵ CFU/ml); and G: glycerol (2%).

FIG. 2 illustrates a flow chart of the microbial method of electrical power generation according to the present invention.

FIG. 3 illustrates a simplified microbial fuel cell apparatus utilizing the electrogenic bacteria and mechanism according to the present invention.

FIG. 4(a) illustrates graphically the voltages generated by S. epidermidis after fermentation. M: rich medium; H: H₂O; BS: S. epidermidis bacteria inoculated (10 CFU/ml); and G: glycerol (2%).

FIG. 4(b) illustrates graphically the voltage difference (ΔmV) generated by P. acnes after fermentation. M: rich medium; H: H₂O; BP: P. acnes bacteria inoculated (10⁵ CFU/ml); and G: glycerol (2%).

### DETAILED DESCRIPTION

Electrogenic bacteria are a heterogeneous group of bacteria, which are not defined by taxonomical or physiological characteristics. The name “electrogenic bacteria” is used for describing these types of bacteria, such as the genus Geobacter and Shewanella, are promising electron generators for MFCs or bioelectrochemical systems (BESs).

To provide a practical solution for microbial power generation, new electrogenic bacteria are identified from human body, and these bacteria are capable of producing electrical power through a complicated biochemical mechanism unique for these bacteria, which is called fermentation. Preferably, the new candidate of electrogenic bacteria is identified herein from natural environment instead of artificially cloned ones.

In suitable embodiments, the newly identified electrogenic bacteria are skin bacteria, which are essentially commensal microorganisms in the human skin microbiome. According to the preferred embodiments of this invention, the skin bacteria Staphylococcus epidermidis is found to be “electrogenic bacteria” for the first time, and their capability of generating electricity and the potential for practical application are validated in the present invention.

The present invention has now been described in accordance with several exemplary embodiments. Staphylococcus epidermidis is a gram-positive bacteria belonging to the genus Staphylococcus. It is a facultative anaerobic bacteria of normal human floram, and it is usually not pathogenic for healthy people. According to preferred embodiments, Staphylococcus epidermidis ATCC12228, Staphylococcus epidermidis RP62A and Propionibacterium acnes Y412MC10 (a skin bacteria for comparison) are used to demonstrate the advantageous effects of the present invention.

Fermentation of S. epidermidis

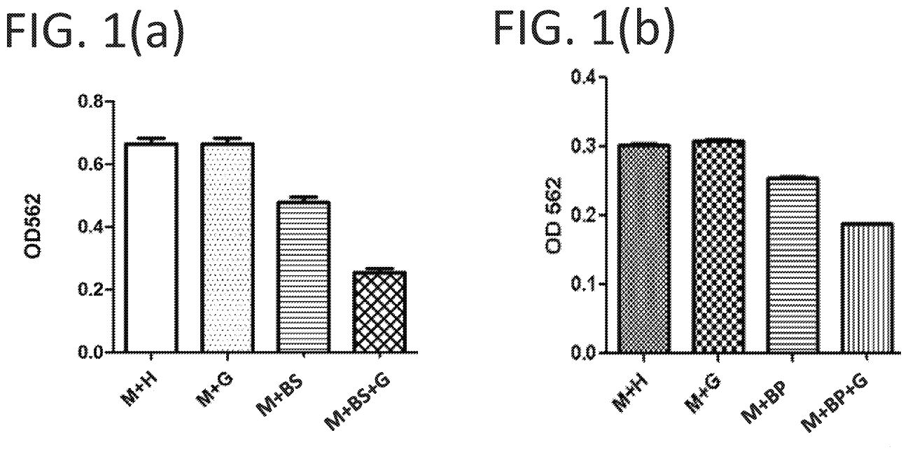

The S. epidermidis (10 CFU/ml) was incubated in rich medium (10 g/L yeast extract, 3 g/L TSB, 2.5 g/L K₂HPO₄, 1.5 g/L KH₂PO₄) in the absence and presence of 2% (w/v) glycerol under anaerobic conditions at 30° C. Rich medium plus 2% (w/v) glycerol without S. epidermidis was included as a control. The 0.002% (w/v) phenol red (Sigma, St. Louis, Mo., USA) in rich medium with 2% glycerol served as an indicator, converting the color of medium from red-orange to yellow when fermentation occurs. Meanwhile, the supernatant of fermented medium was collected respectively, and their absorbance was then read at OD562 nm using a microplate reader. The results were shown in FIG. 1(a), where the read value at OD562 increased as the phenol red converted to yellow.

Fermentation of P. acnes

The P. acnes (10 CFU/ml) was incubated in rich medium (10 g/L yeast extract, 3 g/L TSB, 2.5 g/L K₂HPO₄, 1.5 g/L KH₂PO₄) in the absence and presence of 2% (w/v) glycerol under anaerobic conditions at 30° C. Rich medium plus 2% (w/v) glycerol without P. acnes was included as a control. The 0.002% (w/v) phenol red (Sigma, St. Louis, Mo., USA) in rich medium with 2% glycerol served as an indicator, converting the color of medium from red-orange to yellow when fermentation occurs. Meanwhile, the supernatant of fermented medium was collected respectively, and their absorbance was then read at OD562 nm using a microplate reader. The results were shown in FIG. 1(b), where the read value increased as the phenol red converted to yellow.

Fermentation of bacteria requires appropriate source medium containing suitable fuels. In an embodiment of the present invention, two kinds of source medium S. epidermidis are experimented for fermentation: the rich medium (10 g/L yeast extract, 3 g/L TSB, 2.5 g/L K₂HPO₄, 1.5 g/L KH₂PO₄), and the rich medium (10 g/L yeast extract, 3 g/L TSB, 2.5 g/L K₂HPO₄, 1.5 g/L KH₂PO₄) containing 2% (w/v) glycerol.

As shown in FIG. 1(a), after 48 hours of fermentation, the cultured solution from each group was collected to determine the acidity. These cultures using S. epidermidis as biocatalyst. The result showed that M+BS group and M+BS+G group became acidic as indicated by both phenol red and read value at OD562, because the fermentation of S. epidermidis produced a specific short-chain fatty acid, butyric acid, and may be some butyrate as well. Furthermore, the addition of glycerol (M+BS+G group) apparently enhanced this effect and further lowered the pH of the cultured solution, which means glycerol may favor the fermentation related to S. epidermidis.

On the other hand, the comparator bacteria P. acnes were experimented in the same way as previously described to verify its capability of fermentation. However, as shown in FIG. 1(b), the effects or trends of fermentation (M+BP group and M+BP+G group) were minor.

Identification of Short-Chain Fatty Acids (SCFAs) in the Fermented Media of Bacteria by Nuclear Magnetic Resonance (NMR) Analysis.

To examine the fermentation activity of bacteria, S. epidermidis were incubated in rich medium under anaerobic conditions in the presence of glycerol for 48 hours. Rich media plus either glycerol or S. epidermidis were used as controls. To monitor the fermentation process, these cultures were tested with phenol red, a fermentation indicator, to assess SCFA production as a result of glycerol fermentation. Only media in the culture of S. epidermidis with glycerol turned yellow (more acidic) after incubation, indicating the occurrence of fermentation of S. epidermidis. This finding was further validated quantitatively by measuring the pH values of rich media. The pH values of rich media containing glycerol, S. epidermidis and glycerol plus S. epidermidis were 6.5, 6.4, and 6.0, respectively, following 48 hours of incubation. To identify the SCFAs in the ferments, the S. epidermidis were incubated in rich medium under anaerobic conditions in the presence of ¹³C₃-glycerol (20 g/l) for 48 hours. Supernatants of microbial fermentation in 10% deuterium oxide (D₂O) were subjected to I-D and 2-D ¹³C and ¹H NMR analysis. In addition to ethanol and alanine, four SCFAs (acetic acid, butyric acid, lactic acid, and succinic acid) were detected in the fermented media of S. epidermidis. These four SCFAs, but not ethanol or alanine, were also detectable in the ¹³C₃-glycerol fermented media of S. epidermidis. These results demonstrate that S. epidermidis fermentatively metabolized ¹³C₃-glycerol into SCFAs.

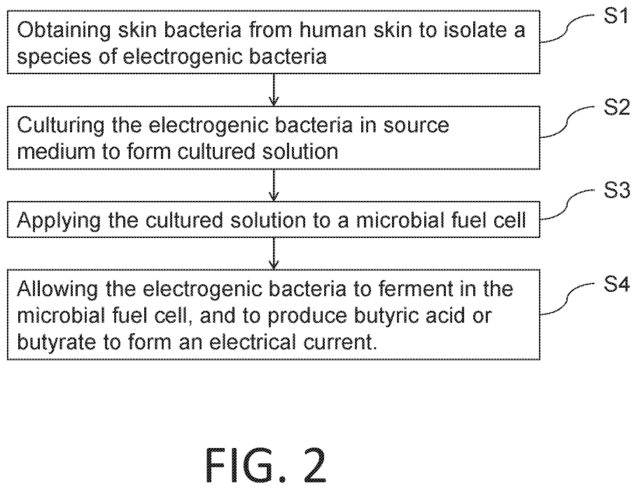

According to the object of developing a novel biological electrogenic system to satisfy the aforementioned demands, a method of electrical power generation is proposed herein. More specifically, the method is a microbial method of electrical power generation. Referring to FIG. 2, the purpose of the step S1 is to acquire a suitable microbe, preferably from human skin microbiome, to develop the biological electron transport system. In one embodiment, various species of skin bacteria are obtained from human skin, and a specific species of electrogenic bacteria is further isolated form those bacteria. Moreover, the source of electrogenic bacteria is not limited, which means the electrogenic bacteria isolated directly from environmental sources, the commercial bacteria cultures, and the acclimatized or artificially manipulated bacterial strains can be used to practice the present invention. Preferably, the electrogenic bacteria can be a species of skin bacteria for the application of miniature medical devices.

Regarding the steps included in the method according to the present invention, step S1 is to obtain a plurality of skin bacteria from a human skin to isolate a species of electrogenic bacteria. However, in an embodiment, the skin bacteria can be purchased from American Type Culture Collection (ATCC) or other bioresource centers. In another embodiment, the skin bacteria can be isolated directly from human skin. For instance, the skin bacteria are isolated from skin fingerprints. According to an embodiment, subjects were invited to participate in fingerprinting, and all subjects were asked not to wash their hands before pressing their fingerprints. Then, fingerprints of fingers (index, middle, and ring fingers) are pressed onto the surfaces of agar plates composed of rich medium (10 ml, containing 10 g/l yeast extract, 5 g/l TSB, 2.5 g/l K₂HPO₄ and 1.5 g/l KH₂PO₄) to select single colonies of microorganism which create inhibition zone. Then, the sequence analysis of 16S rRNA genes of these colonies was performed to identify the microorganisms in fingerprints. In short, the selected single colonies are picked up by sterile toothpicks and boiled at 100° C. for DNA extraction to obtain genetic sequence information. The 16S rRNA gene sequences were analyzed using the basic local alignment search tool (BLASTn).

After a suitable species of electrogenic bacteria are isolated or obtained, these electrogenic bacteria may be maintained, amplified or acclimatized for the following usages. When a bacterial electrogenic system is about to be set, the step S2 is performed, it means the electrogenic bacteria are cultured in source medium to grow. As the electrogenic bacteria grow, the biological processes (such as heterotrophic metabolism, fermentation, and respiration, etc.) and the resultant products make the source medium to form the cultured solution. In one preferred embodiment, the electrogenic bacteria tend to fermentate to generate at least one SCFA, and the primary products/metabolites are butyric acid, any kinds of butyrate, or both the butyric acid and butyrate. During these processes, protons are generated and transferred in the cultured solution, which drives the formation of electrical current.

While the electrogenic bacteria cultured in the source medium are growing, the step 3 is preferably performed to set up a specific microbial fuel cell to make use of the energy derived from the active electron transporting process. Therefore, the cultured solution along with the growing bacteria are appropriately applied to a microbial fuel cell. Furthermore, in addition to the cultured solution itself more ingredients in favor of the specific fermentation can be added into the cultured solution at this stage, to improve the fermentation performance.

Once the biological materials are ready for generating electricity, the step S4 is performed, allowing the electrogenic bacteria to ferment in the microbial fuel cell, and to produce SCFAs, particularly the butyric acid or butyrate, to form electrical current.

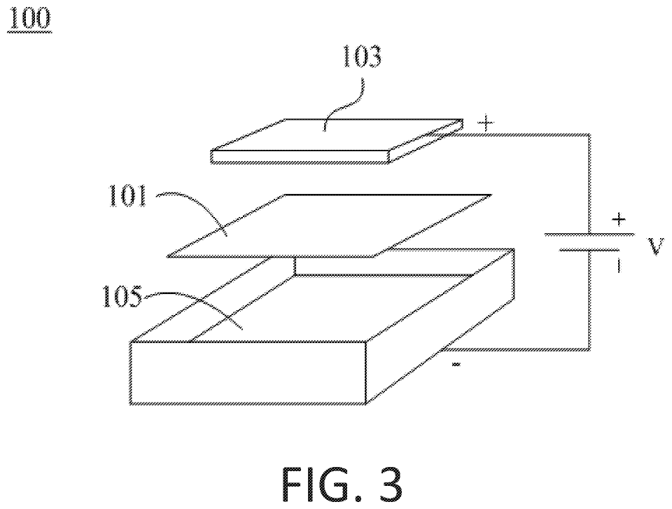

According to the purpose of medical device application, a proof-of-concept apparatus is provided herein. As shown in FIG. 3, an exemplary apparatus 100 based on a microbial fuel cell is constructed by an anode 103, a proton exchange membrane 101 and a cathode 105. The apparatus and process using S. epidermidis as an electrogenic bacteria to generate power includes the following steps: (a) culturing S. epidermidis in the source medium to ferment and to form a cultured solution; (b) applying the cultured solution to the apparatus (microbial fuel cell); and (c) allowing the S. epidermidis to further ferment at the anode (+) of the apparatus, and the fermentation process produces butyric acid, butyrate or both butyric acid and butyrate, among which the protons are generated to form an electrical current.

Preferably, for constructing an economic and dexterous apparatus, the anode of the apparatus is a carbon felt, and the cathode is a carbon cloth. Practically, the cultured solution with fermenting S. epidermidis was loaded to the anode; and the cultured solution then infiltrated into the carbon felt. According to a preferred embodiment of the present invention, at least 10 mL cultured solution should be loaded to the carbon felt for sufficient infiltration, which allowed the cultured solution to contact the proton exchange membrane to ensure the protons generated by S. epidermidis at anode could pass through the proton exchange membrane and finally arrive at the cathode, which is preferably made of a carbon cloth, to form the electrical current.

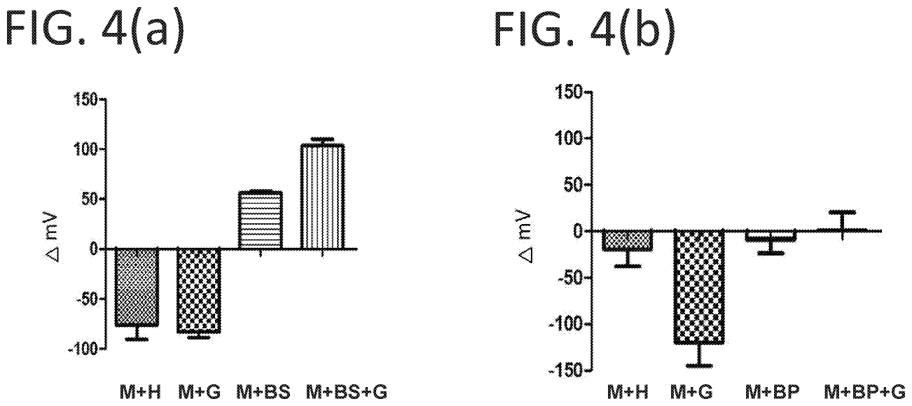

To further measure the electrical current generated from this apparatus 100, a power meter was electrically connected to the cathode 105 and the anode 103 of the apparatus 100, and the voltage difference of each group of the exemplary embodiments listed previously, i.e., M+H, M+G, M+BS and M+BS+G groups, are determined. The measurement results are shown in FIG. 4(a). On the other hand, the voltage difference of the comparator bacteria P. acnes were also determined in the same way. The measurement results are shown in FIG. 4(b). Overall, as shown in FIG. 4(a), with the comparison of P. acnes, the S. epidermidis was validated to generate electrical current, and the voltage difference ranged from 30 mV to 150 mV, and preferably ranged from 50 mV to 110 mV. However, the P. acnes had no similar effects or trends. Apparently, the S. epidermidis was validated as an excellent electrogenic bacteria species, demonstrating its potential to be applied for technological development of MFCs.

1. A microbial method of electrical power generation, comprising:
(a) obtaining a plurality species of skin bacteria from a human skin to isolate a species of electrogenic bacteria;
(b) culturing the electrogenic bacteria in a source medium to form a cultured solution;
(c) applying the cultured solution to a microbial fuel cell; and
(d) allowing the electrogenic bacteria to ferment in the microbial fuel cell, and to produce butyric acid or butyrate to form an electrical current.

2. The method as claimed in claim 1, wherein the electrogenic bacteria are Staphylococcus epidermidis.

3. The method as claimed in claim 1, wherein the cultured solution is applied to the anode of the microbial fuel cell for fermentation.

4. The method as claimed in claim 1, wherein the voltage of the electrical current ranges from 30 mV to 150 mV.

5. An apparatus of electrical power generation, comprising an anode configured to hold a cultured solution containing a species of electrogenic bacteria, allowing the electrogenic bacteria cultured in the cultured solution to produces a least one short-chain fatty acid;
a proton exchange membrane provided for being contacted with the cultured solution containing the electrogenic bacteria; and
a cathode;
wherein the electrogenic bacteria is cultured to ferment and thereby to produce a plurality of protons to pass through the proton exchange membrane and then arrive at the cathode, and thus the plurality of protons are transferred from the anode to the cathode to form an electrical current.

6. The apparatus as claimed in claim 5, wherein the anode is a carbon felt.

7. The apparatus as claimed in claim 5, wherein the cathode is a carbon cloth.

8. The apparatus as claimed in claim 5, wherein the at least one short-chain fatty acid is butyric acid or butyrate.

9. The apparatus as claimed in claim 5, wherein the electrogenic bacteria are Staphylococcus epidermidis.

10. The apparatus as claimed in claim 5, wherein the electrical voltage of the electrical current ranges from 30 mV to 150 mV.
